(19) United States
(12) Patent Application Publication
(10) Pub. No.: US 2024/0141391 A1
(43) Pub. Date: May 2, 2024

(54) METHODS FOR THE PRODUCTION OF A BACTERIA-DERIVED POWDER

(71) Applicant: SUPERBREWED FOOD INC., Newcastle, DE (US)

(72) Inventors: Pradeep Munasinghe, Wilmington, DE (US); Aharon M. Eyal, Jerusalem (IL); Bryan P. Tracy, Wilmington, DE (US)

(21) Appl. No.: 18/384,898

(22) Filed: Oct. 30, 2023

Related U.S. Application Data
(60) Provisional application No. 63/421,243, filed on Nov. 1, 2022.

(51) Int. Cl. C12P 1/04; C12N 1/20

(52) U.S. Cl. CPC C12P 1/04; C12N 1/20; C12R 2001/01

(57) ABSTRACT

Producing a composition comprising cells of at least one bacterium, where at least 90% of the composition is in the form of a free flowing powder, by providing a drum dryer comprising two spaced-apart rotatable cylinders, with a gap of width G inches and a length of L inches therebetween; providing a fermentation broth comprising cells of at least one bacterium in a liquid suspension at a total solid content C percent weight of no greater than about 15% wt of the total weight of the fermentation broth; and introducing the fermentation broth into said drum dryer at a rate of W Kg per hour of no greater than 14×[(G×L)/C] to provide the composition.

### FIELD

The invention, in at least some embodiments, relates to production of a free-flowing powder derived from bacterial cells.

### BACKGROUND

Production of a dry powder from bacterial cells generally involves use of a drum dryer. However, known drying methods commonly result in formation of elongated clumps (ribbons) of bacterial material on the sides of the surface of the drum, which are mainly attributed to insufficient scraping of material off the drum. Multiple layers of such material may then build up on the drum over several cycles of operation of the drum dryer prior to being scraped off, resulting in the material being burnt, providing a burnt taste and/or smell to the powder produced and subsequently to any product produced from such powder.

There is thus an unmet need for a method of production of a free-flowing dry powder from bacterial cells in which the dried bacterial cells are cleanly scraped off the surface of the drum, resulting in a free-flowing dry powder which is substantially devoid of burnt material.

### SUMMARY

According to an aspect of some embodiments of the invention there is provided a method of producing a composition comprising cells of at least one bacterium, wherein at least 90% of the composition is in the form of a free flowing powder, the method comprising providing a drum dryer comprising two spaced-apart rotatable cylinders, with a gap of width G inches and a length of L inches therebetween; providing a fermentation broth comprising cells of at least one bacterium in a liquid suspension at a total solid content C percent weight of no greater than about 15% wt of the total weight of the fermentation broth; and introducing the fermentation broth into said drum dryer at a rate of W Kg per hour of no greater than 14×[(G×L)/C] Kg per square inch per hour to provide the composition.

### DETAILED DESCRIPTION

It is known in the food and drug industries to use a drum dryer to reduce the moisture content of a feedstock during a manufacturing process using steam or hot air. A drum dryer generally comprises two spaced-apart rotatable cylinders, with a gap (also referred to as a nip) between the cylinders.

The inventors have surprisingly found that a free-flowing powder which is substantially devoid of burnt material may be produced from a fermentation broth comprising bacterial cells by careful selection of the drying parameters during use of a drum dryer. The parameters are selected such that the rate of flow (Kg per hour) of said fermentation broth through the nip plane is no greater than 14 times the area of the nip (square inches) divided by the total solid content percentage weight of the fermentation broth. The rate of flow is therefore controlled by selection of the area of the gap, based on the gap width and length, and by the total solid content of the fermentation broth entering the gap, which is in turn controlled by the solid content of the fermentation broth feed to the dryer and the feed rate.

The inventors have found that a rate of flow of greater than 14 causes too much dry mass to pass through the nip, such that ribbons are formed, resulting in burnt material on the drum surface. A rate of flow of no greater than 14 kg/sq inch/hour, more preferably no greater than 10 kg/sq inch/hour is therefore used in the present method.

The particulars shown herein are by way of example and for purposes of illustrative discussion of the various embodiments of the invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the invention. At the very least, and not as an attempt to limit the application of the doctrine of equivalents to the scope of the claims, each numerical parameter should be construed in light of the number of significant digits and ordinary rounding approaches.

As used herein, the term “free-flowing powder” refers to a powder

As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the invention, there is provided a method for producing a composition comprising cells of at least one bacterium, wherein at least about 90% of the composition is in the form of a free flowing powder, the method comprising
a. providing a drum dryer comprising two spaced-apart rotatable cylinders, with a gap of width G inches and a length of L inches therebetween;
b. providing a fermentation broth comprising cells of at least one bacterium in a liquid suspension at a total solid content C percent weight of no greater than about 15% wt of the total weight of the fermentation broth; and
c. introducing said fermentation broth into said drum dryer at a rate of W Kg per hour of no greater than 14×[(G×L)/C] to provide said composition.

According to some embodiments, about 90%, about 91%, about 92%, about 93%, about 94%, about 95%, about 96%, about 97%, about 98%, about 99% or about 100% of the composition is in the form of a free flowing powder.

According to some embodiments, length L is in the range of from about 10 to about 240 inches, such as about 10 inches, about 20 inches, about 30 inches, about 40 inches, about 50 inches, about 60 inches, about 70 inches, about 80 inches, about 90 inches, about 100 inches, about 110 inches, about 120 inches, about 130 inches, about 140 inches, about 150 inches, about 160 inches, about 170 inches, about 180 inches, about 190 inches, about 200 inches, about 210 inches, about 220 inches, about 230 inches or about 240 inches. According to an embodiment for a small scale drum dryer, length L is about 20 inches. According to an embodiment for an industrial dryer, length L is about 240 inches.

According to some embodiments, width G is in the range of from about 0.010 to about 0.015 inches, such as 0.010, 0.012, 0.013, 0.014 or 0.015 inches.

According to some embodiments, the fermentation broth comprises cells of one strain of bacteria, two strains of bacteria, three strains of bacteria or more than three strains of bacteria.

According to some embodiments, total solid content C is from about 9% wt to 15% wt, such as about 9% wt, about 10% wt, about 11% wt, about 12% wt, about 13% wt, about 14% wt or about 15% m of the total weight of the fermentation broth.

According to some embodiments, rate W is no greater than about 14×[(G×L)/C] Kg per hour, such as about [(G×L)/C] Kg per hour, about 2×[(G×L)/C] Kg per hour, about 3×[(G×L)/C] Kg Kg per hour, about 4×[(G×L)/C] Kg per hour, about 5×[(G×L)/C] Kg per hour, about 6×[(G×L)/C] Kg per hour, about 7×[(G×L)/C] Kg per hour, about 8×[(G×L)/C] Kg per hour, about 9×[(G×L)/C] Kg per hour, about 10×[(G×L)/C] Kg per hour, about 11×[(G×L)/C] Kg per hour, about 12×[(G×L)/C] Kg per hour, about 13×[(G×L)/C] Kg per hour or 14×[(G×L)/C] Kg per hour.

According to some embodiments, rate W is in the range of from about 10 to about 300 Kg per hour, such as about 10 Kg/hour, about 20 Kg/hour, about 30 Kg/hour, about 40 Kg/hour, about 50 Kg/hour, about 3560 Kg/hour, about 70 Kg/hour, about 80 Kg/hour, about 90 Kg/hour, about 100 Kg/hour, about 110 Kg/hour, about 120 Kg/hour, about 130 Kg/hour, about 140 Kg/hour, about 150 Kg/hour, about 160 Kg/hour, about 170 Kg/hour, about 180 Kg/hour, about 190 Kg/hour, about 200 Kg/hour, about 210 Kg/hour, about 220 Kg/hour, about 230 Kg/hour, about 240 Kg/hour, about 250 Kg/hour, about 260 Kg/hour, about 270 Kg/hour, about 280 Kg/hour, about 290 Kg/hour or about 300 Kg/hour.

According to some embodiments, a rate of introduction of the fermentation broth into the drum dryer is no greater than about 10×[(G×L)/C] Kg per hour, such as about [(G×L)/C] Kg per hour, about 2×[(G×L)/C] Kg per hour, about 3×[(G×L)/C] Kg per hour, about 4×[(G×L)/C] Kg per hour, about 5×[(G×L)/C] Kg per hour, about 6×[(G×L)/C] Kg per hour, about 7×[(G×L)/C] Kg per hour, about 8×[(G×L)/C] Kg per hour, about 9×[(G×L)/C] Kg per hour, or about 10×[(G×L)/C] Kg per hour.

According to some embodiments, the composition is generated at a rate of from about 5 to about 50 Kg/hour, such as about 5 Kg/hour, about 10 Kg/hour, about 15 Kg/hour, about 20 Kg/hour, about 25 Kg/hour, about 30 Kg/hour, about 35 Kg/hour, about 40 Kg/hour, about 45 Kg/hour or about 50 Kg/hour.

According to some embodiments, the method further comprises heating the drum dryer to provide a surface temperature of each of the rotatable cylinders in a range of from about 100° C. to about 200° C., such as about 100° C., about 110° C., about 120° C., about 130° C., about 140° C., about 150° C., about 160° C., about 170° C., about 180° C., about 190° C. or about 200° C.

According to some such embodiments, the drum dryer is operated to provide a surface temperature of the rotatable cylinders in a range of from about 150° C. to about 160° C., such as about 150° C., about 151° C., about 152° C., about 153° C., about 154° C., about 155° C., about 156° C., about 157° C., about 158° C., about 159° C. or about 160° C.

According to some embodiments, the drum dryer is operated at a steam pressure of at least 40 pounds per square in gauge (psig). According to some such embodiments, the drum dryer is operated at a steam pressure of from about 40 to about 75 psig, such as about 40 psig, about 45 psig, about 50 psig, about 55 psig, about 60 psig, about 65 psig, about 70 psig or about 75 psig.

According to some embodiments, each of the rotatable cylinders comprises a plating layer selected from the group consisting of chrome, stainless steel and combinations thereof. According to some such embodiments, each of the rotatable cylinders comprises a chrome plating layer.

According to some embodiments, the rotatable cylinders are rotated at a speed of from about 5 to about 12 revolutions per minute (rpm), such as about 5 rpm, about 6 rpm, about 7 rpm, about 8 rpm, about 9 rpm, about 10 rpm, about 11 rpm or about 12 rpm. According to some such embodiments, the rotatable cylinders are rotated at a speed of about 9.6 rpm.

According to some embodiments, each of the rotatable cylinders has a diameter of from about 10 to about 75 inches, such as about 10 inches, about 12 inches, about 15 inches, about 20 inches, about 25 inches, about 30 inches, about 35 inches, about 40 inches, about 45 inches, about 50 inches, about 55 inches, about 60 inches, about 65 inches, about 70 inches, about 72 inches or about 75 inches. According to an embodiment, the drum dryer is a small-scale dryer, wherein each of the rotatable cylinders has a diameter of about 12 inches. According to an embodiment, the drum dryer is a commercial-scale dryer, wherein each of the rotatable cylinders has a diameter of about 72 inches.

According to some embodiments, the composition has a moisture content of less than about 4 wt %, such as about 3.9 wt %, 3.5 wt %, 3 wt %, 2.5 wt %, 2 wt %, 1.5 wt %, lwt %, 0.5 wt % or about Owt %. According to some such embodiments, the method further comprises introducing energy into the dryer to provide a residual product moisture content of less than about 4 wt %.

According to some embodiments, at least about 90% of the cells of the at least one bacterium in the composition are in intact form, such as about 90%, about 91%, about 92%, about 93%, about 94%, about 95%, about 96%, about 97%, about 98%, about 99% or about 100% of the cells.

According to some embodiments, the method further comprises heating the concentrated fermentation broth to a temperature of from about 40° C. to about 55° C., such as about 40° C., about 41° C., about 42° C., about 43° C., about 44° C., about 45° C., about 46° C., about 47° C., about 48° C., about 49° C., about 50° C., about 51° C., about 52° C., about 53° C., about 54° C. or about 55° C., prior to introducing into said drum dryer.

What is claimed is:

1. A method of producing a composition comprising cells of at least one bacterium, wherein at least 90% of the composition is in the form of a free flowing powder, the method comprising
a. providing a drum dryer comprising two spaced-apart rotatable cylinders, with a gap of width G inches and a length of L inches therebetween;
b. providing a fermentation broth comprising cells of at least one bacterium in a liquid suspension at a total solid content C percent weight of no greater than about 15% wt of the total weight of the fermentation broth; and
c. introducing said fermentation broth into said drum dryer at a rate of W Kg per hour of no greater than 14×[(G×L)/C] to provide said composition.

2. The method of claim 1, wherein a rate of introduction of said fermentation broth into said drum dryer is no greater than 10×[G×L/C] Kg per hour.

3. The method of claim 1, wherein said composition is generated at a rate of from about 5 to about 50 Kg/hour.

4. The method of claim 1, wherein said rate W is from about 10 to about 300 Kg per hour.

5. The method of claim 1, wherein said width G is from about 0.010 to about 0.015 inches.

6. The method of claim 1, further comprising heating said drum dryer to provide a surface temperature of each of said rotatable cylinders of from about 100° C. to about 200° C.

7. The method of claim 1, wherein said drum dryer is operated at a steam pressure of at least 40 psig.

8. The method of claim 7, wherein said drum dryer is operated at a steam pressure of from about 40 to about 75 psig.

9. The method of claim 8, wherein said drum dryer is operated to provide a surface temperature of said rotatable cylinders of from about 150 to about 160° C.

10. The method of claim 1, wherein each of said rotatable cylinders comprises a plating layer selected from the group consisting of chrome and stainless steel.

11. The method of claim 10, wherein each of said rotatable cylinders comprises a chrome plating layer.

12. The method of claim 1, wherein said rotatable cylinders are rotated at a speed of from about 5 to about 12 revolutions per minute (rpm).

13. The method of claim 12, wherein said rotatable cylinders are rotated at a speed of about 9.6 rpm.

14. The method of claim 1, wherein each said rotatable cylinders have a diameter of about 12 inches.

15. The method of claim 1, wherein said composition has a moisture content of less than about 4 wt %.

16. The method of claim 1, wherein at least 90% of said cells of said at least one bacterium in said composition are in intact form.

17. The method of claim 1, further comprising heating said concentrated fermentation broth to a temperature of from about 40° C. to about 55° C. prior to introducing into said drum dryer.

18. The method of claim 1, further comprising introducing energy into the dryer to provide a residual product moisture content of less than about 4 wt %.
